(19) United States
(12) Patent Application Publication
(10) Pub. No.: US 2025/0032040 A1
(43) Pub. Date: Jan. 30, 2025

(54) System for Monitoring Microcirculation of a Patient

(71) Applicant: Bard Access Systems, Inc., Salt Lake City, UT (US)

(72) Inventors: Stefan Josef Fellner, Salt Lake City, UT (US); Steffan Sowards, Salt Lake City, UT (US); Amelia Marie Smith, Rochester, MI (US)

(21) Appl. No.: 18/226,082

(22) Filed: Jul. 25, 2023

(51) Int. Cl. A61B 5/00

(52) U.S. Cl. CPC A61B 5/412; A61B 5/4839; A61B 5/6804; A61B 5/6832; A61B 5/6892; A61B 5/746

(57) ABSTRACT

Microcirculation assessment systems and methods for determining a systemic response of a patient which may be related to sepsis. A microcirculation assessment device coupled with a patient obtains a microcirculation assessment of the patient. System logic applies an algorithm to the microcirculation assessment to indirectly determine the systemic response. Artificial intelligence and/or machine learning logic collects microcirculation assessment data and corresponding actual systemic responses to define the algorithm that correlates microcirculation assessment with systemic response. System logic may recommend a therapy based the systemic response. System logic may also govern the operation of therapy equipment to deliver a therapy. The microcirculation assessment device may utilize any suitable technology to obtain the microcirculation assessment, such as ultrasound, video capillaroscopy, or thermal imaging, for example. The microcirculation assessment device may be a wearable device or incorporated into hospital bed. Therapy may include body temperature modulation, oxygenation, or infusate delivery.

### BACKGROUND

Sepsis is the body's overwhelming and life-threatening response to infection that can lead to tissue damage, organ failure, and death. Like strokes or heart attacks, sepsis is a medical emergency that requires rapid diagnosis and treatment. Sepsis must be treated quickly and efficiently as soon as healthcare providers suspect it. If it isn't recognized and treated quickly, sepsis can progress to severe sepsis and then to septic shock. It is reported that the chance of sepsis progressing to severe sepsis and septic shock, causing death, rises by 4% to 9% for every hour treatment is delayed. Septic shock is related to hypotension causing a decrease on blood flow to organs causing them to shut down.

Disclosed herein are systems and methods that monitor systemic responses that are associated with sepsis, thereby providing early detection of sepsis and enabling rapid treatment.

### SUMMARY

Disclosed herein is a medical system that, according to some embodiments, includes a microcirculation assessment (MCA) device configured to generate microcirculation data pertaining to a defined location of a patient. The medical system further includes a system module having a console coupled with the MCA device, where the console includes a processor and a memory having logic stored thereon that, when executed by the processor performs operations that include (i) receiving the microcirculation data from the MCA device, (ii) determining a microcirculation assessment from the microcirculation data, and (iii) determining a systemic response of the patient based on the microcirculation assessment.

In some embodiments, systemic response is related to a sepsis condition of the patient.

In some embodiments, the console is wirelessly coupled with the MCA device.

In some embodiments, the MCA device is configured to generate microcirculation data via skin temperature sensing, infrared or near infrared thermal imaging, transcutaneous oxygen measurement, laser doppler flowmetry, dark field imaging, orthogonal polarization spectral imaging, ultrasound imaging, video capillaroscopy, micro-dialysis, or any combination thereof. In some embodiments, the defined location of a patient includes a finger, a nasal tissue, or a sublingual tissue.

In some embodiments, the MCA device is incorporated into a hospital bed. In some embodiments, the MCA device is a wearable device, and in some embodiments, the MCA device is incorporated into an article of clothing.

In some embodiments, a patient contact surface of the MCA device includes an adhesive configured to secure the patient contact surface to the patient.

In some embodiments, the operations further include providing a notification when the systemic response exceeds a defined threshold.

In some embodiments, the operations further include providing a therapy notification based on the systemic response, the therapy notification including a recommended therapy for treating the patient.

In some embodiments, the medical system further includes a display coupled with the console, and the operations further include exhibiting a microcirculation assessment image on the display.

In some embodiments, the medical system further includes a therapy delivery system coupled with the console, where the operations further include initiating the therapy delivery system when the systemic response exceeds a defined threshold. In some embodiments, a therapy of the therapy delivery system includes at least one modulating a body temperature, providing oxygen, or delivering an infusate. In some embodiments, the operations further include adjusting the therapy based on the systemic response.

Also disclosed herein is a system method of determining a systemic response of a patient that, according to some embodiments, includes determining a microcirculation assessment of the patient via a microcirculation assessment (MCA) device that utilizes skin temperature sensing, infrared or near infrared thermal imaging, transcutaneous oxygen measurement, laser doppler flowmetry, dark field imaging, orthogonal polarization spectral imaging, ultrasound imaging, ultrasound doppler, video capillaroscopy, micro-dialysis, or any combination thereof. The system method further includes determining the systemic response based on the microcirculation assessment.

In some embodiments of the system method, determining the systemic response based on the microcirculation assessment includes (i) collecting a plurality of systemic response identification data sets, where each data set includes a microcirculation assessment of a representative patient via the MCA device and an actual systemic response of the representative patient acquired independently from the microcirculation assessment; (ii) defining an algorithm that correlates the actual systemic response with the microcirculation assessment across the plurality of data sets; and (iii) performing the algorithm on the microcirculation assessment of the patient to identify the systemic response.

In some embodiments of the system method, each data set further includes patient data for the representative patient that include one or more of weight, age, sex, or a race, and defining the algorithm includes correlating the actual systemic response with the microcirculation assessment together with the patient data across the plurality of data sets.

In some embodiments, the system method further includes initiating therapy equipment of the system to provide a therapy to the patient based on the microcirculation assessment.

In some embodiments, providing the therapy includes (i) collecting a subset of the plurality of data sets, where each data set of the subset includes the microcirculation assessment of the representative patient, the actual systemic response of the representative patient, and a therapy delivered to the representative patient, and (ii) defining the algorithm to include a correlation of the therapy with the microcirculation assessment. In such embodiments, the system method further includes performing the algorithm on the microcirculation assessment to define the therapy.

These and other features of the concepts provided herein will become more apparent to those of skill in the art in view of the accompanying drawings and following description, which describe particular embodiments of such concepts in greater detail.

### DRAWINGS

FIG. 1 illustrates a medical system configured to determine a systemic response of a patient, in accordance with some embodiments.

FIG. 2 illustrates an exemplary table of data utilized by artificial intelligence logic of the system of FIG. 1 to define an algorithm that correlates a microcirculation assessment with an actual systemic response, in accordance with some embodiments.

FIG. 3 illustrates a block diagram of a system method of determining the system response, in accordance with some embodiments.

FIG. 4 illustrates second embodiment of a microcirculation assessment device of the system of FIG. 1, in accordance with some embodiments.

FIG. 5 illustrates third embodiment of a microcirculation assessment device of the system of FIG. 1, in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise. For example, the “systemic response” a used herein may include a systemic response of one type or a combination of systemic responses of multiple types.

The phrases “connected to,” “coupled with,” and “in communication with” refer to any form of interaction between two or more entities, including but not limited to physical, mechanical, electrical, magnetic, electromagnetic, fluid, wireless, and thermal interaction. Two components may be coupled with each other even though they are not in direct contact or communication with each other. For example, two components may be coupled with each other through an intermediate component.

The term “logic” may be representative of hardware, firmware or software that is configured to perform one or more functions. As hardware, the term logic may refer to or include circuitry having data processing and/or storage functionality. Examples of such circuitry may include, but are not limited or restricted to a hardware processor (e.g., microprocessor, one or more processor cores, a digital signal processor, a programmable gate array, a microcontroller, an application specific integrated circuit “ASIC”, etc.), a semiconductor memory, or combinatorial elements.

Additionally, or in the alternative, the term logic may refer to or include software such as one or more processes, one or more instances, Application Programming Interface(s) (API), subroutine(s), function(s), applet(s), servlet(s), routine(s), source code, object code, shared library/dynamic link library (dll), or even one or more instructions. This software may be stored in any type of a suitable non-transitory storage medium, or transitory storage medium (e.g., electrical, optical, acoustical or other form of propagated signals such as carrier waves, infrared signals, or digital signals). Examples of a non-transitory storage medium may include, but are not limited or restricted to a programmable circuit; non-persistent storage such as volatile memory (e.g., any type of random access memory “RAM”); or persistent storage such as non-volatile memory (e.g., read-only memory “ROM”, power-backed RAM, flash memory, phase-change memory, etc.), a solid-state drive, hard disk drive, an optical disc drive, or a portable memory device. As firmware, the logic may be stored in persistent storage.

Unless defined otherwise, all technical and scientific terms used herein have the same meaning as commonly understood by those of ordinary skill in the art.

Any methods disclosed herein include one or more steps or actions for performing the described method. The method steps and/or actions may be interchanged with one another. In other words, unless a specific order of steps or actions is required for proper operation of the embodiment, the order and/or use of specific steps and/or actions may be modified. Moreover, sub-routines or only a portion of a method described herein may be a separate method within the scope of this disclosure. Stated otherwise, some methods may include only a portion of the steps described in a more detailed method. Additionally, all embodiments disclosed herein are combinable and/or interchangeable unless stated otherwise or such combination or interchange would be contrary to the stated operability of either embodiment.

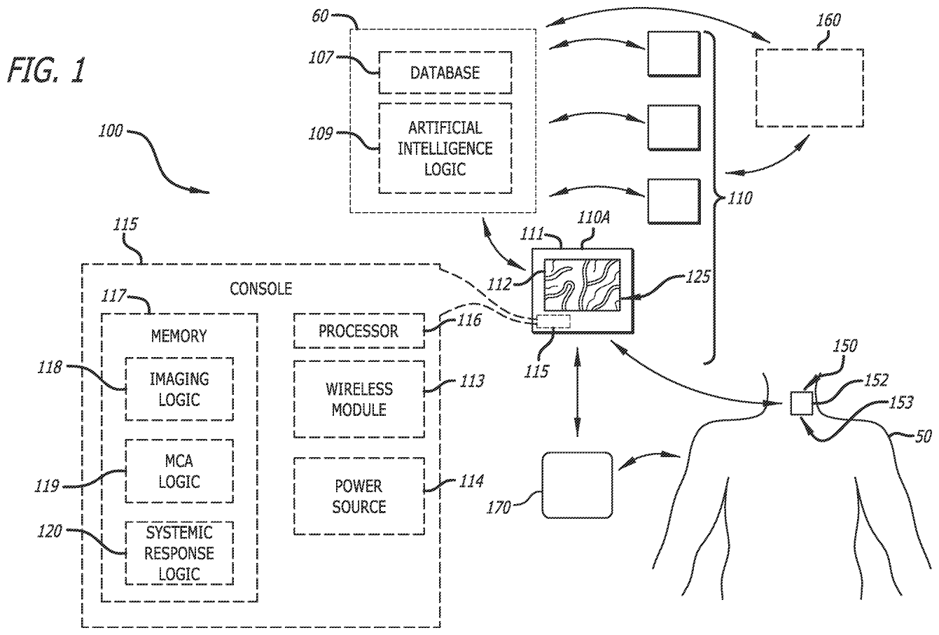

FIG. 1 illustrates a medical system 100 configured to obtain a microcirculation assessment of a patient and determine a systemic response of the patient from the microcirculation assessment, according to some embodiments. In some embodiments or instances of use, the medical system 100 may a determine a systemic response of a patient undergoing a sepsis. As such, the medical system 100 may be configured to indirectly monitor a sepsis condition of the patient. As may be recognized by one of ordinary skill, a sepsis event is just one patient condition that can cause a systemic response suitable for determination via a microcirculation assessment. As such, the medical system 100 may be configured to monitor any patient condition, currently known or unknown, that causes a systemic response suitable for determination via a microcirculation assessment.

The medical system 100 generally includes a plurality of microcirculation assessment (MCA) systems 110. In some embodiments, each of the MCA systems 110 is communicatively coupled (e.g., wirelessly coupled) with an external computing device 60. The external computing device 60 may be any suitable computing device, such as a network server, for example. In some embodiments, the external computing device 60 may be coupled with an electronic medical record (EMR) system 160. An artificial intelligence (AI) logic 109 and a database 107 are stored in memory (e.g., a non-transitory computer-readable medium) of the external computing device 60. The AI logic 109 performs AI and/or machine learning operations on the data stored in the database 107 as further described below. In some embodiments, the MCA systems 110 and the external computing device 60 may be communicatively coupled with the electronic medical record system (EMR) 160.

The MCA system 110A represents any one of the MCA systems 110. The MCA system 110A includes an MCA device 150. The MCA device 150 is generally configured to obtain the microcirculation assessment of the patient 50. The microcirculation assessment includes MCA data pertaining to any one of blood flow rate calculations, perfused vessel calculations, a vessel density, a micro-vasculature flow index, a vessel diameter, or any combination thereof. The microcirculation assessment can be indicative of a systemic response, such as changes in a blood flow, perfusion, or central venous pressure, for example resulting from the sepsis event. In some embodiments, the MCA system 110A may be configured to obtain other vital data from the patient, such as blood pressure, body temperature, pulse rate, and the like in addition to the microcirculation assessment.

The MCA device 150 may be configured to obtain the microcirculation assessment via skin temperature sensing, infrared or near infrared thermal imaging, transcutaneous oxygen measurement, laser doppler flowmetry, dark field imaging, orthogonal polarization spectral imaging, ultrasound imaging, ultrasound doppler, video capillaroscopy, micro-dialysis, or any combination thereof. The MCA device 150 may be configured to obtain the microcirculation assessment at a defined location of the patient 50, such as the neck, for example, as illustrated in FIG. 1. In other embodiments, the MCA device 150 may be configured to obtain the microcirculation assessment at other defined patient locations.

The MCA device 150 may be configured to obtain the microcirculation assessment automatically or in response to an action of a user (e.g., a clinician or the patient), such as pushing a button, for example. In some embodiments, the user may position the MCA device 150 on the patient at the defined location to obtain a single microcirculation assessment. Similarly, the user may repeatedly position the MCA device 150 on the patient at the defined location to repeatedly obtain a single microcirculation assessments as desired. In some embodiments, the user may repeatedly position the MCA device 150 on the patient at the defined location to repeatedly obtain a microcirculation assessments according to a defined schedule so as to obtain a trend of the microcirculation assessments. In other embodiments, the MCA device 150 may be configured to be secured to the patient so that the MCA device 150 may automatically obtain the microcirculation assessments continuously or repeatedly, such as according to a defined schedule. In some embodiments or deployments, the MCA device 150 may be secured to the patient over an extend period of time, such as several days, for example.

According to one embodiment as shown in FIG. 1, the MCA device 150 may include a patch 152. In some embodiments, the patch 152 may include an adhesive 153 disposed across an underside (i.e., a patient contact surface) of the patch 152. The patch 152 may also be secured to patient via a band or a strap.

According to one embodiment, the patch 152 may be incorporated into a hospital bed. For example, the patch 152 may be placed on a mattress such that the patient contact surface is facing upward. In such embodiments, the patch 152 may be sandwiched between the mattress and the patient so that the patch 152 is secured in operative contact with the patient. According to another embodiment, the patch 152 may be incorporated in an article of clothing, such as a sock, a sleeve, an underwear, a glove, or a watch, for example. According to another embodiment, the MCA device 150 may be incorporated into a medical device configured insertion into the patient, such as a probe, a stylet or a catheter, for example.

The MCA system 110A includes a system module 111 having a console 115 and the MCA device 150 is communicatively coupled with the console 115, such as via a wireless connection as illustrated or a wired connection. In the illustrated embodiment, the console 115 includes a processor (or multiple processors) 116 that executes logic stored in a memory 117 (e.g., a non-transitory computer-readable medium), such as the imaging logic 118, the MCA logic 119, and the systemic response logic 120. A power source 114 of the console 115 may include an external source (i.e., a facility power source) or a battery, such as a rechargeable battery, for example. As such, the MCA system 110A may be used within a healthcare facility or away from the healthcare facility, such as at a home or an office, for example. The console 115 may include a wireless module 113 to facilitate wireless communication between the MCA device 150 and the system module 111 and/or between the system module 110A and the external computing device 60 or the EMR system 160. In some embodiments, the system module 111 and the MCA device 150 may be combined to form a single unit.

In some embodiments, MCA system 110A may include (or be coupled with) a display 112 (e.g., a graphical user interface) configured to display notification information and/or receive input from the user. In some embodiments, the imaging logic 118 may create and a cause an MCA image 125 to be exhibited on the display 112. The MCA image 125 may enable the user (e.g., a trained clinician) to visually detect an anomaly or change in the microcirculation assessment, such as a change in a blood vessel diameter or shape for example. In some embodiments, the MCA image 125 may be configured to indicate conditions or measurements of the microcirculation assessment. For example, the MCA image 125 may include colors to indicate conditions of specific aspects of the microcirculation assessment. By way of one specific example, a blood vessel may (i) be exhibited in a red color when blood flow through the blood vessel is in a normal range and (ii) be exhibited in a black color when the blood flow through the blood vessel is in decreased range. By way of another specific example, a background of the MCA image 125 may (i) be exhibited in a green color when the microcirculation assessment is in a normal range and (ii) be exhibited in a yellow color when the microcirculation assessment is in an abnormal range.

The MCA logic 119 generally governs the operation of the MCA device 150 to obtain the microcirculation assessment or assessments, as described above. In some embodiments, the MCA logic 119 may be configured to provide a notification to the user. For example, the MCA logic 119 may generate an alert when the MCA device is unable to obtain the microcirculation assessment, such as when the MCA device 150 is decoupled from the patient 50, for example. The MCA logic 119 may be configured to receive raw data from the MCA device 150 and convert the raw data into the MCA data (described above). In some embodiments, the MCA logic 119 may be configured to compare an instant microcirculation assessment with a known or previously obtained microcirculation assessment.

The image logic 118 may receive the MCA data from the MCA logic 119 for portrayal on the display 112. The systemic response logic 120 is configured to receive the MCA data from the MCA logic 119 and determine or identify the systemic response (described above) based on the MCA data. In some embodiments, the systemic response logic 120 may be configured to determine a trend or change in the systemic response overtime.

The systemic response logic 120 may be configured to provide a notification to the user based on the systemic response. By way of one example, the notification may include an alert (visible and/or audible) when the systemic response exceeds one or more defined thresholds, such as a central venous pressure below a defined limit. By way of another example, the notification may include an alert when a trend or change of the systemic response exceeds a threshold, such as a change (e.g., decrease) in the central venous pressure exceeding a defined limit.

The systemic response logic 120 may be configured to suggest or recommend by way of notification a treatment therapy for the patient 50 based on the systemic response. Patients undergoing a sepsis event have been shown to benefit from therapies that include body temperature modulation, oxygenation, and/or medication (e.g., an IV infusate), for example. Medications may include antibiotics and/or vasodilators, for example. As such, the systemic response logic 120 may recommend a treatment therapy for the patient 50 based on the systemic response that includes temperature modulation, oxygenation, medication, or any combination thereof.

In some embodiments, the MCA system 110A may include or be coupled with medical equipment 170 configured to provide a therapy to the patient 50. The medical equipment 170 may be any equipment suitable for treating the patient 50. For example, in the case of a patient undergoing a sepsis event, the medical equipment 170 may include a body temperature management system, an oxygen source, an infusion pump, or any combination thereof. The medical equipment 170 is effectively coupled with the patient 50 so as to provide the therapy. The MCA system 110A may be coupled with medical equipment 170 so that the MCA system 110A may govern the operation of the medical equipment 170 in accordance with providing the therapy. In some embodiments, the MCA system 110A may initiate the therapy only upon approval input from the user. As such, the MCA system 110A when coupled with the medical equipment 170 may be configured to treat a sepsis condition of the patient 50.

According to one embodiment, the body temperature management system may include a targeted temperature management system utilizing one or more thermal pads applied to the skin surface of the patient to warm or cool the patient. In such an embodiment, the patch 152 or the MCA device 150 generally may be attached to or incorporated into a thermal pad.

The MCA system 110A may be configured to utilize machine learning and/or artificial intelligence techniques to determine the systemic response from the microcirculation assessment. As stated above, the MCA system 110A may be coupled with the external computing device 60 having a database 107 and running the AI logic 109.

The AI logic 109 is generally configured to define a relationship or correlation between the microcirculation assessment and actual systemic response. The AI logic 109 utilizes AI and/or machine learning techniques to define the correlation based on a collection of data sets and stored in the data base 107. In some embodiments, the AI and/or machine learning techniques may include statistical operations, such as linear regression, for example. The AI logic 109 may then define the algorithm based on the correlation, where the algorithm is configured to determine a systemic response for an instant patient based on the microcirculation assessment of the instant patient. The systemic response logic 120 is configured to acquire the microcirculation assessment of the instant patient and apply the algorithm to the microcirculation assessment to determine systemic response of the instant patient.

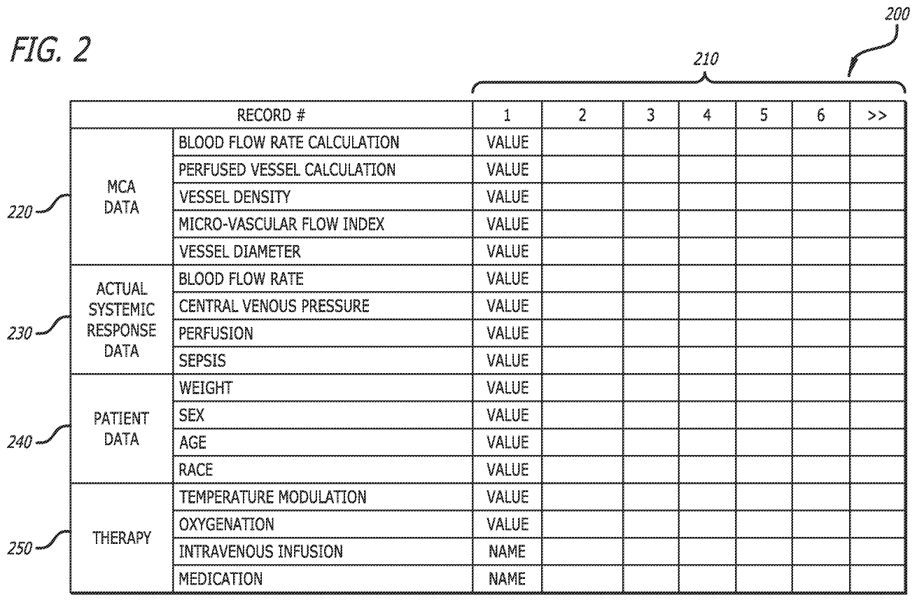

FIG. 2 illustrates an exemplary table 200 of the database 107, according to some embodiments. The table 200 includes a number (e.g., 10, 100, 1000, or more) data sets (or records) 210 as collected from the plurality of the MCA systems 110 where each data set 210 may include data pertaining to a microcirculation assessment event for a patient. The data table 200 includes MCA data 220 as obtained by the MCA device 150 and actual systemic response data 230 obtained independently from the microcirculation assessment. In some instances of a microcirculation assessment event, such as a sepsis event, actual systemic response data may be acquired directly from the patient utilizing suitable methodologies and techniques for such. For example, a central venous pressure may be directly measured via a central catheter. Similarly, sepsis data may be obtained via blood testing. It is noted that each data set 210 may not include all of the MCA data 220 and/or the actual systemic response data 230 shown in the table 200. In other words, a data set 210 may include only a subset of the MCA data 220 and/or the actual systemic response data 230 indicated in the table 200.

In some embodiments, the actual systemic response data 230 may be input into the MCA system 110 by a clinician via the display 112. In such embodiments, the MCA system 110 may transmit the MCA data 220 and the actual systemic response data 230 to the external computing device 60 as a data set for processing by the AI logic 109. In other embodiments, the actual systemic response data 230 may be included in an electronic medical record (EMR) for the patient and in such embodiments, the external computing device 60 may acquire the systemic response data 230 from the EMR system 160.

In some embodiments, though not required, the table 200 may include patient data 240. The patient data 240 may include data, such as weight, sex, age, or race, for example. Of course, the patient data 240 may not be limited to the patient data shown. For example, additional patient data (not shown) may include blood pressure, medication, disease, or any other medical conditions of the patient. In some embodiments, the patient data 240 may include only a subset of the patient data shown. In some embodiments, the patient data 240 may be recorded in the EMR for the patient, and as such, the external computing device 60 may acquire the patient data 240 from the EMR system 160. In such embodiments, the AI logic 109 define a relationship or correlation between the microcirculation assessment data 220 together with the patient data 240 and the actual systemic response data 230, and the AI logic 109 define the algorithm accordingly.

The AI logic 109 may continue to collect or receive additional data sets from the plurality of MCA systems 110 and/or the EMR system 160 to refine the algorithm, e.g., increase an accuracy or statistical confidence of the algorithm. In some embodiments, the AI logic 109 may regularly (e.g., continually) receive additional data sets.

With the algorithm defined by the AI logic 109, the systemic response logic 120 may utilize the algorithm to determine the systemic response from the microcirculation assessment. In other words, the systemic response logic 120 may apply the algorithm to the MCA data acquired by the MCA device 150 for the patient 50 to determine the systemic response for the patient 50. As the MCA system 110A is communicatively coupled with the external computing device 60, the MCA system 110A may acquire the algorithm from the external computing device 60.

In some embodiments, the AI logic 109 may be configured to define a relationship or correlation between the actual systemic response and a delivered therapy. In some cases of a microcirculation assessment event, the clinician may deliver a therapy to the patient to treat the patient condition, e.g., sepsis. In such cases, the clinician may record in the EMR the therapy delivered and the patient response to the therapy, such as the effectiveness of the therapy in relieving the patient condition, for example. As such, the table 200 may include therapy data 250. The AI logic 109 may then further define the algorithm to include the correlation between the actual systemic response and the delivered therapy so that the systemic response logic 120, via application of the algorithm, is configured to determine the recommended the therapy for the patient based on the microcirculation assessment.

By way of one example, the systemic response logic 120 may determine that the systemic response based on the microcirculation assessment indicates severe sepsis. In such a case, infusion therapy may be a typical therapy for treating the severe sepsis. The clinician may deliver the infusion therapy and record that the infusion therapy was effective in treating the sepsis. The external computing device 60 may acquire the microcirculation assessment from the MCA system 110A. The external computing device 60 may also acquire the actual systemic response and the therapy information/data from the EMR system 160. As result of this example, the table 200 may include a data set 210 that includes the MCA data 220, the corresponding actual systemic response data 230, and the therapy data 250 (i.e., the infusion therapy data including the effectiveness of the infusion therapy in treating the sepsis).

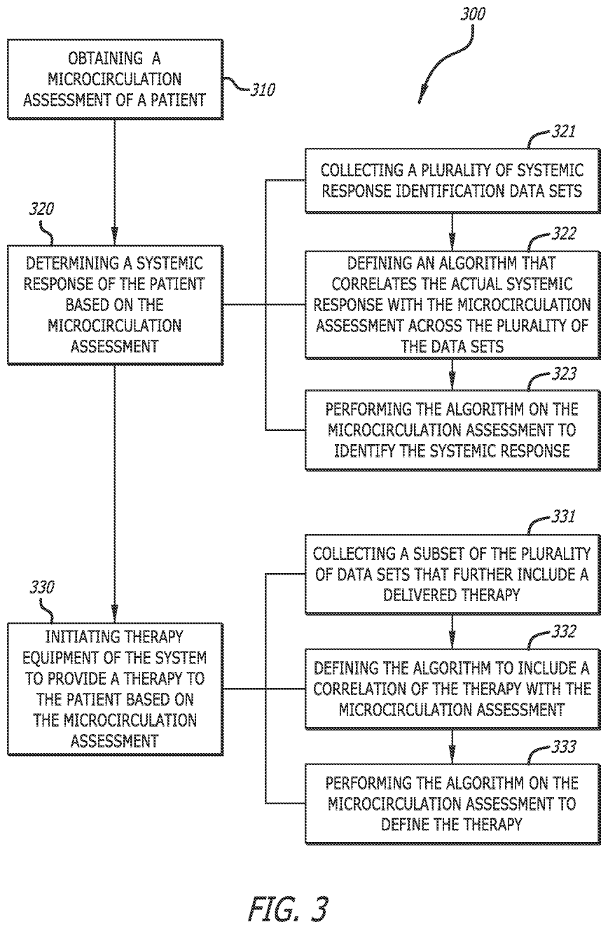

FIG. 3 is a block diagram of a system method 300 of determining a systemic response of a patient, as performed by the medical system 100 that, according to some embodiments, includes all or any subset of the following steps, actions, or processes. The system method 300 includes obtaining a microcirculation assessment of a patient (block 310). Obtaining the microcirculation assessment is performed by a microcirculation assessment (MCA) device that utilizes skin temperature sensing, infrared or near infrared thermal imaging, transcutaneous oxygen measurement, laser doppler flowmetry, dark field imaging, orthogonal polarization spectral imaging, ultrasound imaging, ultrasound doppler, video capillaroscopy, micro-dialysis, or any combination thereof. The system method 300 further includes determining a systemic response of the patient based on the microcirculation assessment (block 320). The systemic response may include all or any subset of blood flow rate, perfusion, or central venous pressure, including changes thereof.

The system method 300 may further include collecting a plurality of systemic response identification data sets (block 321), where each data set includes a microcirculation assessment of a representative patient via the MCA device and an actual systemic response of the representative patient acquired independently from the microcirculation assessment. The system method 300 may further include defining an algorithm that correlates the actual systemic response with the microcirculation assessment across the plurality of the data sets (block 322), and performing the algorithm on the microcirculation assessment to identify the systemic response (block 323).

In some embodiments of the system method 300, each data set further includes patient data for the representative patient, such as weight, age, sex and/or race. As such, the system method 300 may further including defining the algorithm to correlate the actual systemic response with the microcirculation assessment together with the patient data across the plurality of data sets.

The system method 300 may further include initiating therapy equipment of the system to provide a therapy to the patient based on the microcirculation assessment (block 330). In some embodiments, the system method 300 may further include collecting a subset of the plurality of data sets that further include a delivered therapy (block 331), i.e., each data set of the subset includes the microcirculation assessment of the representative patient, the actual systemic response of the representative patient, and a therapy delivered to the representative patient. The system method 300 may further include defining the algorithm to include a correlation of the therapy with the microcirculation assessment (block 332). In such embodiments, the system method 300 may further include performing the algorithm on the microcirculation assessment to define the therapy (block 333).

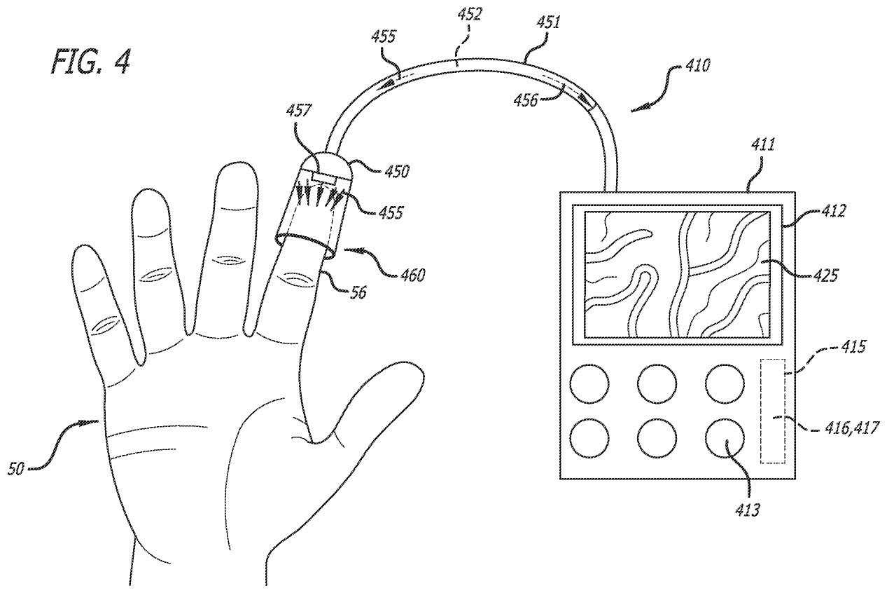

FIG. 4 illustrates another embodiment of an MCA system 410 that can, in certain respects, resemble components of the MCA system 110A described in connection with FIG. 1. It will be appreciated that all the illustrated embodiments may have analogous features. Accordingly, like features are designated with like reference numerals, with the leading digits incremented to “4.” For instance, the MCA device is designated as “150” in FIG. 1, and an analogous MCA device is designated as “450” in FIG. 4. Relevant disclosure set forth above regarding similarly identified features thus may not be repeated hereafter. Moreover, specific features of the MCA system 410 and related components shown in FIG. 1 may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the MCA system 410 of FIG. 4. Any suitable combination of the features, and variations of the same, described with respect to the MCA system 110A and components illustrated in FIG. 1 can be employed with the MCA system 410 and components of FIG. 4, and vice versa. This pattern of disclosure applies equally to further embodiments depicted in subsequent figures and described hereafter.

The MCA system 410 generally includes the MCA device 450 and the system module 411. The system module 411 includes a display 412 for exhibiting the MCA image 425. The system module 411 may be sized and shaped to enable hand holding of the system module 411. Buttons 413 provide input and control of the MCA system 410. The console 415 of the MCA system 410 may include a light source 416 and/or a photo detector 417. A connecting member 451 couples the MCA device 450 with the system module 411. In some embodiments, the connecting member 451 may include a number (e.g., 1, 2, 3, or more) of optical fibers 452 extending therealong. The connecting member 451 may include a sufficient length (e.g., 2-4 feet) to enable placement of the system module 411 in clothing pocket or purse, for example, during use.

The MCA system 410 is configured to obtain a microcirculation assessment of a pad portion or other portion of a finger 56 of the patient. The MCA device 450 may be configured to obtain a microcirculation assessment via blood flow rate calculations, vessel identification, transcutaneous oxygen measurement, microvascular flow index, total vessel density, portion of perfused vessels, laser doppler flowmetry, dark field imaging, orthogonal polarization spectral imaging, ultrasound imaging, ultrasound doppler, video capillaroscopy, or any combination thereof. The MCA device 450 may include attachment device 460, such as a clamp, a clip, or a cuff, for example, configured to secure the MCA device 450 to the finger 456.

In some embodiments, one or more of the optical fibers 452 may be configured to transport light 455 from the light source 417 to the MCA device 450 where the light 455 illuminates the finger 56 to enable imaging of the micro-blood vessels of the finger 56. Similarly, image light 456 emanating from the finger 56 in response to the light 455 passes through a lens 457 upon entering the one or more other optical fibers 452 that are configured to transport the image light 456 from the MCA device 450 to the photodetector 417.

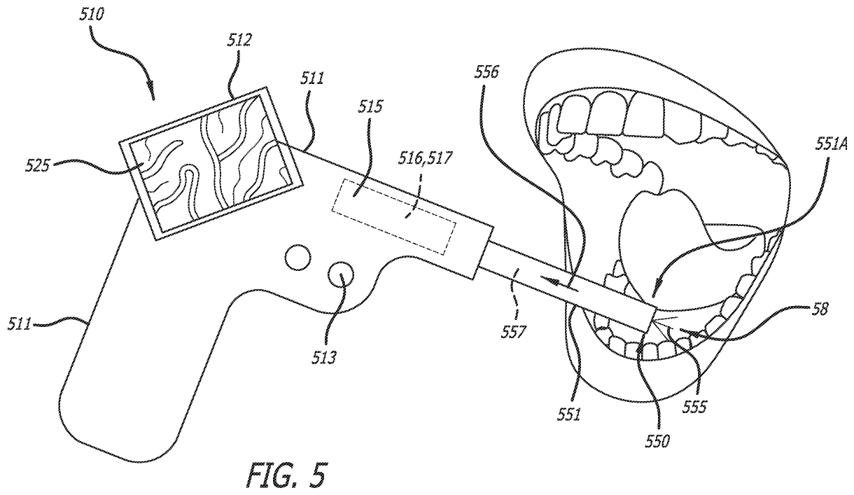

FIG. 5 illustrates another embodiment of an MCA system 510 that generally includes the MCA device 550 and the system module 511 combined into a single unit. The system module 511 includes a display 512 for exhibiting the MCA image 525. The system module 511 may be sized and shaped to enable hand holding of the system module 511. The MCA system 510 may include buttons 513 to enable input and control of the MCA system 510. The console 515 of the MCA system 510 may include a light source 516 (e.g., an LED) and/or a photo detector 517.

The MCA system 510 is configured to obtain a microcirculation assessment of a sublingual tissue 58 of the patient according to one embodiment. Alternatively or in addition to, the MCA system 510 may be configured to obtain a microcirculation assessment of a nasal tissue (not shown) according to another embodiment. The MCA device 550 may be configured to obtain a microcirculation assessment via blood flow rate calculations, vessel identification, transcutaneous oxygen measurement, microvascular flow index, total vessel density, portion of perfused vessels, laser doppler flowmetry, dark field imaging, orthogonal polarization spectral imaging, ultrasound, video capillaroscopy, or any combination thereof.

In some embodiments, the extending member 551 may be configured transport light 555 from the light source 517 to the distal end 551A where the light 555 illuminates the sublingual tissue 58 to enable imaging of the micro-blood vessels of the sublingual tissue 58. Similarly, the extending member 551 may be configured transport image light 556 emanating from the sublingual tissue 58 from the distal end 551A to the photo detector 517. In some embodiments, the extending member 551 may include a lens 557. In some embodiments, the extending member 551 may include a number (e.g., 1, 2, 3, or more) of optical fibers 552 extending therealong between the system module 511 and a distal end 551A to facilitate transporting the light 555 or the image light 556 along the extending member 551.

Although that the MCA system 510 is configured for obtaining the microcirculation assessment from the sublingual tissue 58 and or nasal tissue, one of ordinary skill would appreciate the MCA system 510 may configured to interface with and obtain microcirculation assessments from any other suitable body tissue without deviating from the disclosure above.

The MCA systems 110, 410 and 510 are each configured for deployment with the medical system 100. The medical system 100 may include a plurality of the MCA systems including any number of MCA systems 110, 410, and/or 510.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. A medical system, comprising:
a microcirculation assessment (MCA) device configured to generate microcirculation data pertaining to a defined location of a patient; and
a system module having a console coupled with the MCA device, the console including a processor and a memory having logic stored thereon that, when executed by the processor performs operations that include:
receiving the microcirculation data from the MCA device;
determining a microcirculation assessment from the microcirculation data; and
determining a systemic response of the patient based on the microcirculation assessment.

2. The medical system according to claim 1, wherein the systemic response is related to a sepsis condition of the patient.

3. The medical system according to claim 1, wherein the console is wirelessly coupled with the MCA device.

4. The medical system according to claim 1, wherein the MCA device is configured to generate microcirculation data via skin temperature sensing, infrared or near infrared thermal imaging, transcutaneous oxygen measurement, laser doppler flowmetry, dark field imaging, orthogonal polarization spectral imaging, ultrasound imaging, video capillaroscopy, micro-dialysis, or any combination thereof.

5. The medical system according to claim 1, wherein the defined location of a patient includes a finger, a nasal tissue, or a sublingual tissue.

6. The medical system according to claim 1, wherein the MCA device is incorporated into a hospital bed.

7. The medical system according to claim 1, wherein the MCA device is a wearable device.

8. The medical system according to claim 7, wherein the MCA device is incorporated into an article of clothing.

9. The medical system according to claim 1, wherein a patient contact surface of the MCA device includes an adhesive configured to secure the patient contact surface to the patient.

10. The medical system according to claim 1, wherein the operations further include providing a notification when the systemic response exceeds a defined threshold.

11. The medical system according to claim 1, wherein the operations further include providing a therapy notification based on the systemic response, the therapy notification including a recommended therapy for treating the patient.

12. The medical system according to claim 1, further including a display coupled with the console, the operations further including exhibiting a microcirculation assessment image on the display.

13. The medical system according to claim 1, further comprising a therapy delivery system coupled with the console, the operations further including initiating the therapy delivery system when the systemic response exceeds a defined threshold.

14. The medical system according to claim 12, wherein a therapy of the therapy delivery system includes at least one of modulating body temperature, providing oxygen, or delivering an infusate.

15. The medical system according to claim 12, wherein the operations further include adjusting the therapy based on the systemic response.

16. A system method of determining a systemic response of a patient, comprising:
obtaining a microcirculation assessment of the patient via a microcirculation assessment (MCA) device of a system that utilizes skin temperature sensing, infrared or near infrared thermal imaging, transcutaneous oxygen measurement, laser doppler flowmetry, dark field imaging, orthogonal polarization spectral imaging, ultrasound imaging, video capillaroscopy, micro-dialysis, or any combination thereof; and
determining the systemic response based on the microcirculation assessment.

17. The system method according to claim 16, wherein determining the systemic response based on the microcirculation assessment comprises:
collecting a plurality of microcirculation assessment data sets, wherein each data set includes a microcirculation assessment of a representative patient via the MCA device and an actual systemic response of the representative patient acquired independently from the microcirculation assessment,
defining an algorithm that correlates the actual systemic response with the microcirculation assessment across the plurality of data sets; and
performing the algorithm on the microcirculation assessment of the patient to determine the systemic response.

18. The system method according to claim 17, wherein:
each data set further includes patient data for the representative patient that include one or more of weight, sex, age, or race, and
defining the algorithm includes correlating the actual systemic response with the microcirculation assessment together with the patient data across the plurality of data sets.

19. The system method according to claim 17, further comprising initiating therapy delivery equipment of the system to provide a therapy to the patient based on the systemic response.

20. The system method according to claim 19, wherein providing the therapy includes:
collecting a subset of the plurality of data sets, each data set of the subset including the microcirculation assessment of the representative patient, the actual systemic response of the representative patient, and a therapy delivered to the representative patient;
defining the algorithm to include a correlation of the therapy with the microcirculation assessment; and
performing the algorithm on the microcirculation assessment to define the therapy.
